(19) United States
(12) Patent Application Publication
(10) Pub. No.: US 2026/0263104 A1
(43) Pub. Date: Sep. 10, 2026

(54) DEVICE FOR OSTEO IMPLANTS

(71) Applicant: MERIL HEALTHCARE PVT. LTD, Vapi (IN)

(72) Inventors: VIJAY C. BOSE, Chennai (IN); DARSHAN A. LAD, Bilimora (IN); JIMESH B. GANDHI, Bilimora (IN); PRATIK M. PANCHAL, Vapi (IN); LATHEESH KV, Kannur (IN)

(73) Assignee: MERIL HEALTHCARE PVT LTD, Vapi, GJ (IN)

(21) Appl. No.: 19/491,342

(22) PCT Filed: Jun. 7, 2024
(86) PCT No.: PCT/IN2024/050702

§ 371 Date: Dec. 8, 2025

(30) Foreign Application Priority Data
Jun. 7, 2023 (IN) 202321039118

(51) Int. Cl. A61B 17/32; A61B 17/00

(52) U.S. Cl. CPC A61B 17/32; A61B 2017/00477; A61B 2017/320052

(57) ABSTRACT

The present disclosure relates to a device for defining a bone cavity. The device includes a link member, a reamer and a guide member. The link member includes at least one rotatable coupling mechanism to couple the guide member and the reamer. The reamer and the guide member are coupled to opposite ends of the link member. The reamer is configured to rotate around the pivot of the guide member. The guide member being configured as a pivot point. The guide member includes a head portion and a tail portion. The tail portion of the guide member couples to the implant.

### FIELD OF INVENTION

The present disclosure relates to a device for osteo implants. More specifically, the present disclosure discloses a device to ream a cavity around an osteo implant.

### BACKGROUND OF INVENTION

People suffering from deteriorating hip joints undergo a hip replacement surgery. In the hip replacement surgery, the hip joint is completely or partially replaced by a synthetic implant.

Naturally, the hip joint includes a femur bone (thigh bone) coupled to a pelvic bone via a ball and socket joint. In the hip replacement surgery, to reinforce the hip joint, the femur bone is provided with a stem prosthesis (or implant). A conventional stem prosthesis includes an axially extending stem portion and a neck portion that is disposed at an angle with respect to the stem portion. The stem prosthesis is inserted within the cavity of the femur bone. The neck portion indirectly connects the stem portion to the pelvic bone.

In about 30% of the cases, it is observed that after inserting the stem prosthesis, there exists an undesirable gap between a proximal portion of the stem prosthesis and the femur bone such that the proximal portion of the stem prosthesis is left unsupported. Such gaps, if left unnoticed, are prone to implant failure and/or bone (femur) fracture.

Conventional devices include complex mechanisms and provide limited support to the surrounding of the implanted prosthesis inside the bone cavity.

Therefore, there arises a need for a system that provides adequate support to the surrounding of the implanted stem prosthesis for defining the gap between the implant and the bone and also have simple construction providing ease of use.

### SUMMARY OF INVENTION

Particular embodiments of the present disclosure are described herein below with reference to the accompanying drawings, however, it is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The present disclosure relates to a device for defining a bone cavity including a guide member, a reamer, and a link member. A tail portion of the guide member is configured to be inserted within at least one recess of an implant. Opposite ends of the link member include the guide member and the reamer. The reamer is configured to rotate around the pivot of the guide member. The guide member being configured as a pivot point. The guide member includes a head portion and a tail portion. The tail portion of the guide member couples to the implant.

The foregoing features and other features as well as the advantages of the disclosure will become more apparent from the following detailed description, which proceeds with reference to the accompanying figures.

### BRIEF DESCRIPTION OF DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts an isometric view of a device 100, according to an embodiment of the present disclosure.

FIG. 2 depicts an exploded view of the device 100, according to an embodiment of the disclosure.

FIG. 3 depicts an isometric view of a link member 110 of the device 100, according to an embodiment of the present disclosure.

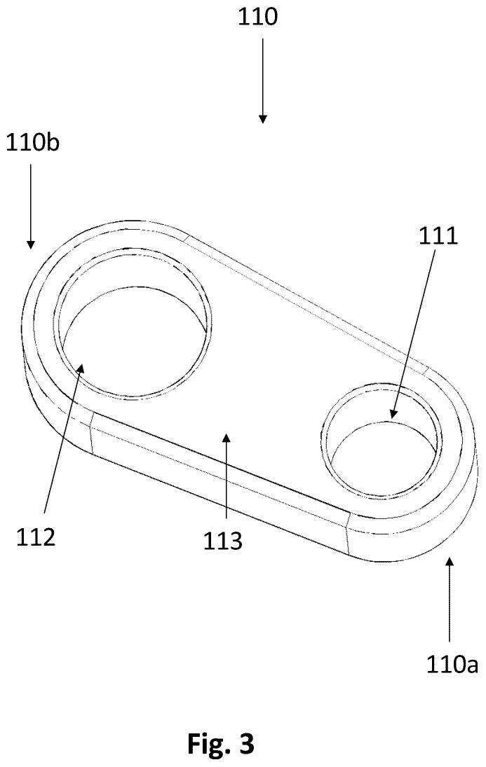

FIG. 4 depicts an isometric view of a reamer 120 of the device 100, according to an embodiment of the present disclosure.

FIG. 5 depicts an isometric view of a guide member 130 of the device 100, according to an embodiment of the present disclosure.

FIG. 6 depicts the device 100 coupled to the implant 300 implanted within the femur bone 200 in accordance with an embodiment of the present disclosure.

FIG. 7 depicts an exemplary flowchart of a method 700 for operating the device 100 according to an embodiment of the present disclosure.

### DETAILED DESCRIPTION OF THE DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

In the context of the present disclosure, a gap refers to the space between a bone and an implant, once the implant is inserted. The term cavity refers to a reshaped or redefined gap in which an augment is inserted. Typically, the dimensions of the cavity correspond to the dimensions of the augment.

The device of the present invention is explained in the context of a femur bone of a patient during a hip surgery. However, it is envisaged that at least some of the embodiments of this disclosure may instead pertain to usage of the device in the context of a humeral implant for shoulder surgery. The configuration of the device in such embodiments may be substantially as described below, although the overall size and shape of the device may be configured to allow it to operate in the humeral bone area instead of a femur. It is also envisaged that at least some embodiments of this invention may instead pertain to usage of the device in the context of a (distal) femoral implant or a tibial implant for knee surgery.

The present disclosure discloses a device used to define a cavity in a bone corresponding to the measurements of an augment to be inserted in the cavity around an osteo implant (or implant).

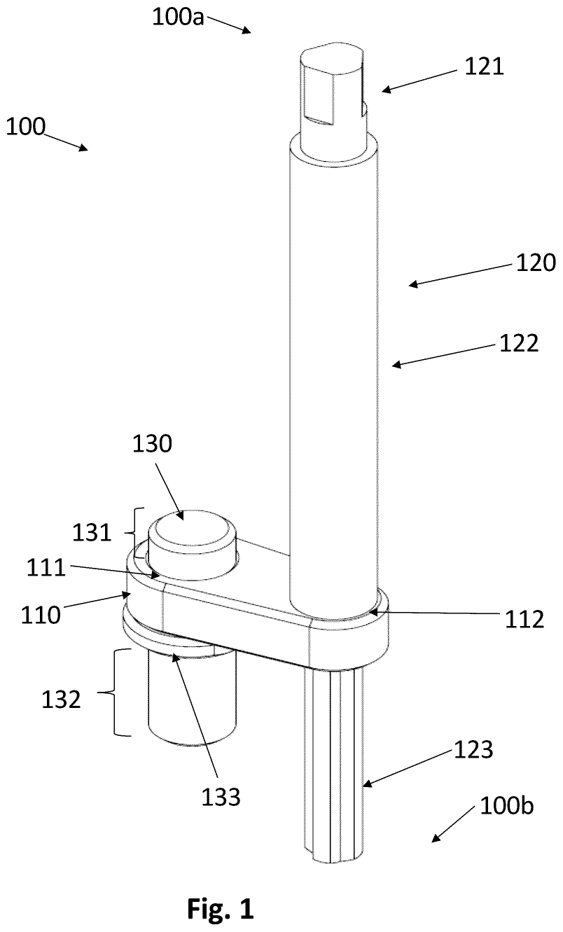

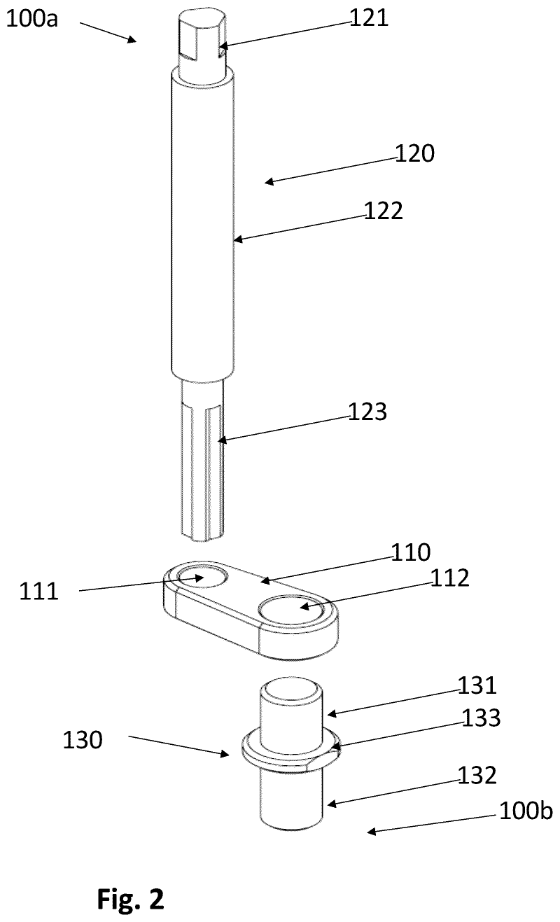

Now referring to figures, FIG. 1 depicts an exemplary embodiment of a device 100 while FIG. 2 depicts an exploded view of the device 100. The device 100 is configured to define a cavity in a femur bone around the implant corresponding to the measurements of an augment. Typically, the gap left inside the femur bone once the implant is inserted, may be non-uniform or vary in dimensions from one individual to another. This gap if not filled, leads to implant failure and/or bone (femur) fracture. Therefore, to reshape or reconfigure the dimensions of the gap according to the dimensions of an augment or other filling means to be placed in the femur bone 200, the device 100 is used. The device 100 is used to perform a milling action in a precise area to reshape or reconfigure the gap defining a cavity in the femur bone 200.

The device 100 includes a proximal side 100a and a distal side 100b. The device 100 includes a link member 110, a reamer 120 and a guide member 130.

The link member 110 includes a first end 110a and a second end 110b. At least one end of the link member 110 includes at least one rotatable coupling mechanism. The rotatable coupling mechanism couples the link member 110 with the reamer 120 and/or the guide member 130. In an embodiment, opposite ends of the link member 110 including the rotatable coupling mechanism, are coupled to the reamer 120 and the guide member 130. The link member 110 can be of any suitable shape such as cuboid, sphere, cylinder, trapezoid, etc. In an embodiment, the link member 110 is of a trapezoid shape with rounded corners. The link member 110 can be made from any suitable biocompatible material such as titanium, CoCr, stainless steel, or any other metal alloys. In an embodiment, the link member 110 is made of SS 17-4 PH.

In an exemplary embodiment depicting the assembly of the link member 110, the reamer 120 and the guide member 130, apertures are provided with the link member 110. The link member 110 includes at least two apertures. In an embodiment, the at least two apertures include a first aperture 111 and a second aperture 112. The first aperture 111 and the second aperture 112 extend from a first face 113 to a second face (not shown) of the link member 110. The first aperture 111 and the second aperture 112 can be positioned at a predefined location along the length of the link member 110. In an embodiment, the first aperture 111 is placed towards the first end 110a of the link member 110 and the second aperture 112 is placed towards the second end 110b of the link member 110. Thus, the first aperture 111 and the second aperture 112 are separated by a predefined distance for milling the bone surrounding the proximal portion of the stem. The predefined distance varies depending upon the diameter of the stem of the implant and the bone anatomy of the patient.

The diameter of the first aperture 111 and the second aperture 112 may be equal or may vary. In an embodiment, the first aperture 111 includes a reduced diameter as compared to the second aperture 112. The diameter of the first aperture 111 is at least 5 mm. The diameter of the second aperture 112 is at least 5 mm. The first aperture 111 and the second aperture 112 may include a smooth, threaded or semi-threaded internal surface. In an embodiment, the first aperture 111 and the second aperture 112 include a smooth internal surface. Optionally, the first aperture 111 and the second aperture 112 may be tapered.

In an exemplary embodiment, the link member 110 can include a length adjustment mechanism to adjust the distance between the first aperture 111 and the second aperture 112. The length adjustment mechanism may provide variable length adjustment options to the surgeon during procedure.

The first aperture 111 of the link member 110 is configured to allow passage of at least a portion of the reamer 120. The second aperture 112 of the link member 110 is configured to allow passage of the guide member 130.

In an embodiment, the first aperture 111 or the second aperture 112 or both include a rotating mechanism. For instance, the first aperture 111 includes the rotating mechanism to facilitate rotation of the reamer 120. The rotating mechanism includes without limitation bushes, bearings, etc. Due to the rotating mechanism, the reamer 120 is configured to be rotatably coupled with the first aperture 111 of the link member 110. In an embodiment, the reamer 120 and the first aperture 111 are coupled using a bearing (not shown) to the link member 110.

Alternately, the first aperture 111 and second aperture 112 may include other constructions that facilitate the coupling of the reamer 120 and the guide member 130 with the link member 110. For example, the apertures include snap-fit or slot-fit slots configured to receive at least a portion of the reamer 120 or the guide member 130.

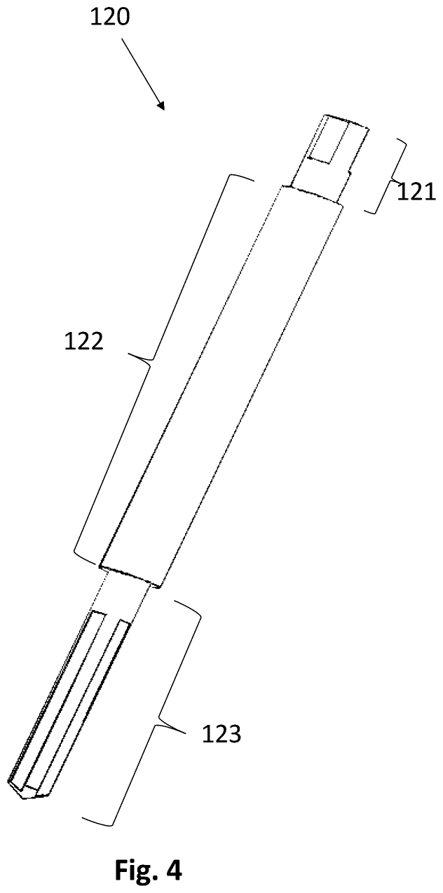

The reamer 120 includes without limitation a cylindrical reamer, a tapered reamer, an expansion reamer, an adjustable reamer, etc. In an embodiment, the reamer 120 is a cylindrical reamer. The reamer 120 includes an adaptor 121, a shank portion 122 and a flute portion 123 (FIG. 4). The top portion of the reamer 120 includes the adaptor 121 while the bottom portion of the reamer 120 includes the flute portion 123 with the shank portion 122 therebetween. The adaptor 121, the shank portion 122 and the flute portion 123 can form an integral unit or can include a coupling mechanism to form the reamer 120. In an embodiment, the reamer 120 is an integral unit including the adaptor 121, the shank portion 122 and the flute portion 123. The reamer 120 can be made from any suitable bio compatible material such as stainless steel, titanium, CoCr, or any other metal alloys.

The shank portion 122 is operatively coupled to the rotating mechanism included inside the first aperture 111 of the link member 110. The said coupling provides rotatability to the reamer 120 w.r.t. to the link member 110.

In an exemplary embodiment, the reamer 120 includes an outer casing coupled to the link member 110 that covers at least a portion of the reamer 120. In an embodiment, the outer casing covers the shank portion 122 of the reamer 120. The casing includes a suitable coupling mechanism such that the reamer 120 can rotate freely inside the casing. The flute portion 123 of the reamer 120 extends from the casing for unrestricted reaming or milling action.

The adaptor 121 of the reamer 120 is coupled to an external rotating mechanism such as a power tool (not shown). The external rotation mechanism provides rotatability to the reamer 120. During rotation of the reamer 120, the link member 110 remains stationary due to the rotational coupling provided between the first aperture 111 and the reamer 120.

The diameter of the flute portion 123 may vary depending upon the width of the cavity to be reamed. In an embodiment, the flute portion 123 may be removably coupled to the shank portion 122. The flute portion 123 of appropriate diameter may be coupled while reaming the femur bone. Alternately, the flute portion 123 may be of uniform diameter or include tapering diameter.

The second aperture 112 of the link member 110 is coupled to the guide member 130 using any suitable mechanism such as snap fit, press fit, etc. In an embodiment, the guide member 130 and the link member 110 are coupled using a press-fit mechanism. The guide member 130 may be of any suitable shape. In an embodiment, the guide member 130 is of a cylindrical shape. The guide member 130 can be made from any suitable bio compatible materials such as stainless steel, titanium, CoCr or any other suitable metal alloy.

The guide member 130 includes a head portion 131 and a tail portion 132. In an embodiment, the guide member 130 is rotatably coupled to the link member 110 via the second aperture 112. The second aperture 112 may include any suitable mechanism to facilitate the rotational coupling with the guide member 130. The mechanism includes for example, a bearing, a bush etc. In an embodiment, the second aperture 112 includes a bearing (not shown). The second aperture 112 acts as a pivot point for the device 100 to rotate around the implant 300 inside the bone cavity.

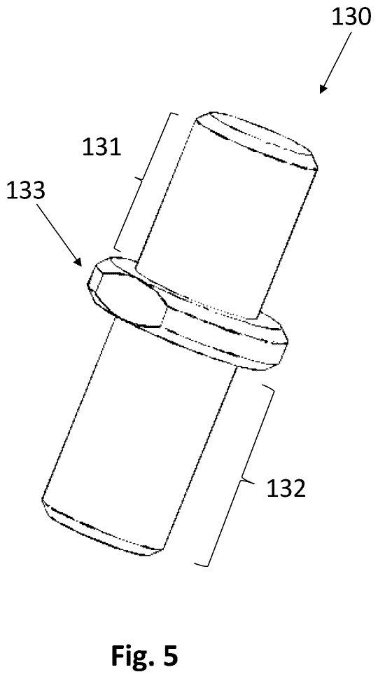

Further, the link member 110 can include a height adjustment mechanism such that the link member 110 is inserted at a desired level on the implant 300. In an embodiment, the height adjustment mechanism includes a stopper 133. The stopper 133 can be placed at any suitable position along the length of the link member 110 (FIG. 5). The stopper 133 opposes further motion of the link member 110 to move beyond the preset length.

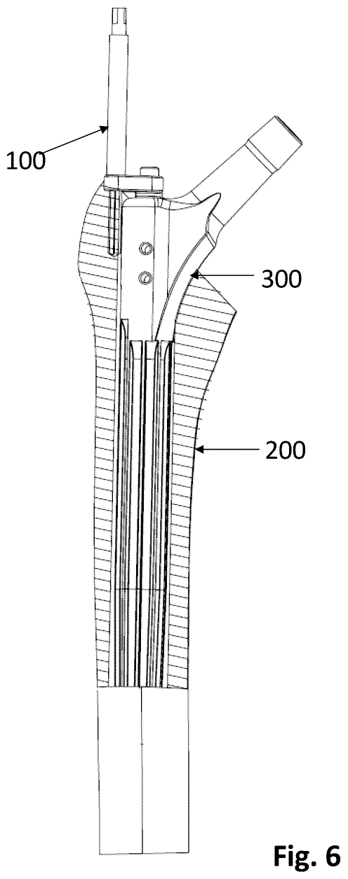

At least a portion of the guide member 130 is configured to couple with an implant 300. In an embodiment, the tail portion 132 of the guide member 130 is coupled with the implant 300 (FIG. 6). The guide member 130 and the implant 300 can be coupled using any suitable method such as snap-fit, press-fit, friction fit, fastening, threaded mechanism, etc. In an embodiment, the guide member 130 and the implant 300 are coupled using a threaded mechanism. In an embodiment, the tail portion 132 of the guide member 130 is configured to be inserted within at least a portion of a recess of the implant 300. As shown in FIG. 6, the guide member 130 is coupled to the recess of the implant 300 implanted inside a bone 200.

FIG. 6 depicts an exemplary embodiment of an assembly of an exemplary implant 300, a femur bone 200 and the device 100. As can be seen, post implantation of the implant 300, the device 100 is mounted on the implant 300 to ream a cavity of dimensions reciprocating the dimensions of an augment to be inserted in the cavity. In an embodiment, an augment of appropriate dimensions is selected by a surgeon depending upon the dimensions of the femur bone. Alternately, an augment of standard dimensions may be used. While FIG. 6 depicts one embodiment of the implant, the device of the present disclosure can be used with other known implants as well.

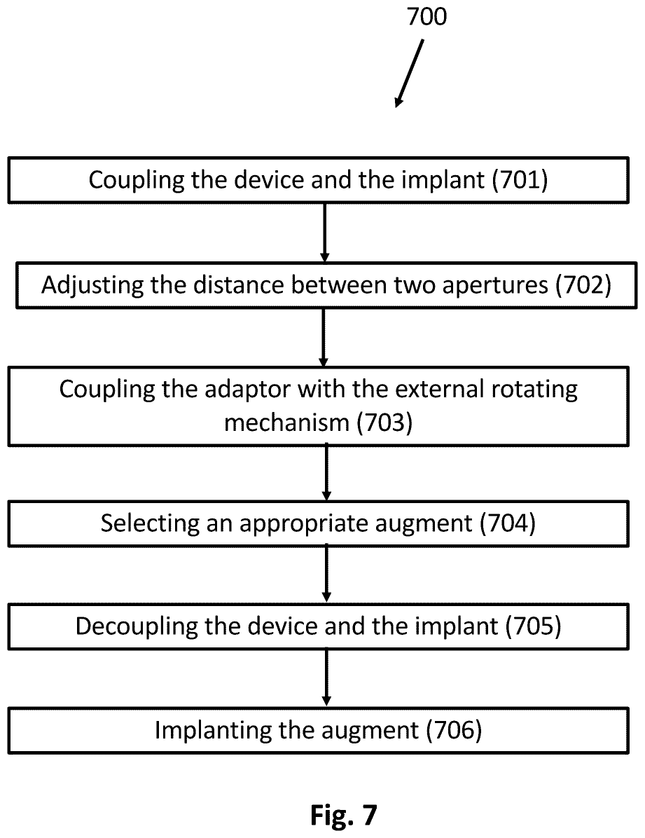

The tail portion 132 of the guide member 130 is inserted within at least a portion of a recess of the implant 300 while the reamer 120 is positioned outside and adjacent the implant. The link member 110 supporting the two, maybe positioned on top of the implant 300. The method of reaming a cavity is described in FIG. 7 below.

FIG. 7 illustrates a flowchart of a method 700 of operating the device 100 for reaming a cavity of desired dimensions according to an embodiment. The device 100 is used to ream a bone, for example, a femur bone 200.

At step 701, the device 100 is coupled to the implant 300 when the surgeon feels the requirement after noticing a gap between the implant 100 and the femur bone 200. The surgeon places the tail portion 132 of the guide member 130 inside the recess of the implant 300.

Step 702 is optionally performed by the surgeon as the link member 110 may or may not include a length adjustment mechanism. After coupling the device 100, the surgeon adjusts the length of the link member 110 such that the flute portion 123 of the reamer 120 is placed in the gap between the implant 300 and the femur bone 200. Initially, a reamer 120 having narrow dimensions may be selected so that it can be easily inserted in the gap between the implant 300 and the bone.

At step 703, the adaptor 121 of the reamer 120 is coupled to an external rotating mechanism such as a power drill. The external rotating mechanism rotates the reamer 120 and the flute portion 123 to activate the reaming action. The surgeon reshapes the gap in the femur bone 200 to form a cavity such that the cavity can be filled by inserting the augment.

At step 704, the surgeon may insert at least one trial augment in the cavity to check and select an appropriate trial augment. The surgeon may repeat this step with trial augments of different thickness by inserting them one after the other till a best fit trial augment is selected. An augment corresponding to the best fit trial augment is selected. By following this procedure and selecting the augment of appropriate dimensions, the cavity is filled properly ensuring the longevity of the implant 300. Thickness of the trial augments can range between 5 mm and 10 mm or specified otherwise.

At step 705, the device 100 and the implant 300 are decoupled by unfastening the fastener (not shown). Further, an appropriate augment is selected.

At step 706, the augment is coupled/inserted inside the cavity. After insertion of the augment, the surgeon couples the augment and the implant 300 using a fastener.

The milling device thus brings lot of simplicity to the complicated procedure and helps in reaming a cavity of required dimensions in which an augment is thereafter inserted to support the stem prosthesis.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. A device (100) for defining a bone cavity comprising:
a link member (110) including at least one rotatable coupling mechanism provided towards at least one end of the link member (110);
a reamer (120); and
a guide member (130) including a head portion (131) and a tail portion (132), the guide member (130) being configured as a pivot point, the tail portion (132) of the guide member (130) being configured to couple with an implant (300);
wherein the reamer (120) and the guide member (130) are coupled to opposite ends of the link member (110); and
wherein the reamer (120) is configured to rotate around the pivot point of the guide member (130).

2. The device (100) as claimed in claim 1, wherein at least one of the link member (110), the reamer (120), and the guide member (130) is made from a bio compatible material.

3. The device (100) as claimed in claim 1, wherein the link member (110) includes a first aperture (111) and a second aperture (112).

4. The device (100) as claimed in claim 3, wherein the first aperture (111) and the second aperture (112) include a rotating mechanism.

5. The device (100) as claimed in claim 4, wherein the rotating mechanism includes one of a bush, or a bearing.

6. The device (100) as claimed in claim 3, wherein the first aperture (111) and the second aperture (112) include a slot-fit or snap-fit slot.

7. The device (100) as claimed in claim 3, wherein the first aperture (111) and the second aperture (112) include one of a smooth, threaded or semi-threaded internal surface.

8. The device (100) as claimed in claim 3, wherein at least one of the first aperture (111) and the second aperture (112) are tapered.

9. The device (100) as claimed in claim 1, wherein the reamer (120) comprises:

10. An adaptor (121), a flute portion (123) and a shank portion (122) there between, wherein the adaptor (121) is coupled to an external rotating mechanism to providing rotatability to the reamer (120).
